(19) United States
(12) Patent Application Publication
(10) Pub. No.: US 2021/0244291 A1
(43) Pub. Date: Aug. 12, 2021

(54) FLUID FLOW RATE DETERMINATIONS USING VELOCITY VECTOR MAPS

(71) Applicant: SCINOVIA, CORP., Sheridan, WY (US)

(72) Inventors: James Bradley Sund, Raleigh, NC (US); David S. Cohen, Chapel Hill, NC (US); Heinrich Ziegler, Dutton (CA)

(21) Appl. No.: 17/245,779

(22) Filed: Apr. 30, 2021

Related U.S. Application Data
(63) Continuation of application No. 15/401,572, filed on Jan. 9, 2017.
Application No. 15/401,572 is a continuation of application No. PCT/US15/39589, filed on Jul. 8, 2015.
(60) Provisional application No. 62/022,189, filed on Jul. 8, 2014.

(51) Int. Cl. A61B 5/026; A61B 5/0285; A61B 5/00; G06T 7/12

(52) U.S. Cl. CPC A61B 5/026; A61B 5/0261; A61B 5/0285; A61B 5/7246; A61B 5/107; G06T 7/12; A61B 5/7278; A61B 5/7425; G06T 2207/30101; A61B 5/0066

(57) ABSTRACT

A method of determining volume flow rate of a bodily fluid in a biological conduit includes determining a cross-sectional area of a biological conduit using a velocity vector map representing moving entities or moving fluid portions in a bodily fluid flowing within the biological conduit, calculating an average speed of the moving entities or moving fluid portions in the bodily fluid flowing across the determined cross-sectional area of the biological conduit, and calculating volume flow rate of the bodily fluid in the biological conduit from the determined cross-sectional area and the calculated instantaneous or average speed. A series of velocity vector maps may be collected over time so as to generate a flow rate profile representing flow rate as a function of time.

### CROSS-REFERENCE TO RELATED APPLICATIONS

This application is a continuation of U.S. Non-Provisional patent application Ser. No. 15/401,572, filed Jan. 9, 2017, titled “FLUID FLOW RATE DETERMINATIONS USING VELOCITY VECTOR MAPS”, which is a continuation of International Patent Application Serial No. PCT/US15/39589, filed Jul. 8, 2015, titled “FLUID FLOW RATE DETERMINATIONS USING VELOCITY VECTOR MAPS, which claims the benefit of priority of U.S. Provisional Patent Application No. 62/022,189, titled “IN TISSUE AND VESSEL, FLUID FLOW RATE MEASUREMENTS,” filed on Jul. 8, 2014, which are all incorporated herein in their entirety by this reference.

### TECHNICAL FIELD

The present disclosure relates to the determination of fluid flow rates using velocity vector maps, for example in living tissue (human, animal, and plant) and fluid vessels such as blood, lymphatic, digestive tract and plant circulation vessels.

### BACKGROUND

Non-invasive medical imaging techniques permit some degree of visualization of internal fluid flows such as that of blood vessels and other biological conduits. For example, laser light creates a speckle response on human, animal, and plant tissue. A speckle response can include complex interference patterns, resulting from refraction, diffraction and reflection phenomena. A dynamic speckle response is related to the blood and other fluid flow activity in tissue, vessels, or plant circulation. Several algorithms are available within the scientific community to interpret light speckle response images; however, none has ever been used to determine calibrated fluid flow rates (FFR), and particularly blood flow rates in human tissue.

Current technologies focus on relative fluid flow or blood flow derived from contrast imaging techniques; however, none of these techniques is currently quantifiable and thus cannot be used to determine actual fluid flow rate (FFR).

### SUMMARY

This summary is provided to introduce in a simplified form, concepts that are subsequently described in detail. This summary is not intended to identify key features or essential features of the claimed subject matter, nor is it to be construed as limiting the scope of the claimed subject matter.

In at least one embodiment, a method of determining volume flow rate, e.g., ml/min, of a bodily fluid in a biological conduit includes: determining a cross-sectional area of a biological conduit using a velocity vector map representing moving entities or moving fluid portions in a bodily fluid flowing within and outside the biological conduit; calculating an average speed of the moving entities or moving fluid portions in the bodily fluid flowing across the determined cross-sectional area of the biological conduit; and calculating volume flow rate of the bodily fluid in and around the biological conduit from the determined cross-sectional area and the calculated average speed.

In at least one example, determining the cross-sectional area of the biological conduit includes determining edges of the biological conduit.

In at least one example, determining edges of the biological conduit includes identifying edges in the velocity vector map.

In at least one example, determining edges of the biological conduit includes determining gradients.

In at least one example, determining edges of the biological conduit includes a user determining the edges viewing a display of the velocity vector map.

In at least one example, determining the cross-sectional area of the biological conduit includes generating a contour map.

In at least one example, determining the cross-sectional area of the biological conduit includes identifying parallel line segments in the contour map.

In at least one example, determining the cross-sectional area of the biological conduit includes determining a width of the biological conduit and assuming the width represents a diameter.

In at least one example, determining the cross-sectional area of the biological conduit includes mathematically identifying borders of the biological conduit and performing orthogonal vector analysis to retrieve a diameter.

In at least one example, the method further includes receiving indication of a user choice of vessel category according to fluid vessel volume.

In at least one example, the method further includes receiving indication of a user choice of vessel category according to biological tissue type.

In at least one example, the method further includes receiving indication of a user choice of vessel category according to anatomical classification.

In at least one example, calculating volume flow rate includes calculating a theoretical fluid flow rate (TFFR) value, and correcting the TFFR value with calibration data from a database, e.g., average human adult male blood flow in a coronary artery branch.

In at least one embodiment, a method of determining volume flow rate of a bodily fluid in a biological conduit in which the flow of the bodily fluid is continuously variable includes: collecting, at a frequency of collection, a series of velocity vector maps over time, the velocity vector maps representing moving entities or moving fluid portions in a bodily fluid flowing within a biological conduit; determining a cross-sectional area of the biological conduit using at least one of the velocity vector maps; calculating an average speed of the moving entities or moving fluid portions in the bodily fluid flowing across the determined cross-sectional area of the biological conduit; and calculating volume flow rate of the bodily fluid in the biological conduit from the determined cross-sectional area and the calculated average speed.

In at least one example, the frequency of collection satisfies a Nyquist criterion for a flow rate profile of the bodily fluid, i.e., imaging sampling rate of movement is at least 2× the acceleration of movement.

In at least one example, the frequency of collection is greater than a Nyquist criterion for a flow rate profile of the bodily fluid.

In at least one example, the method further includes calculating a volume flow rate profile.

In at least one example, calculating an average speed includes calculating an average speed from multiple velocity vector maps of the series of velocity vector maps.

In at least one example, collecting a series of velocity vector maps over time includes collecting a series of velocity vector maps representing blood flow in a bodily organ over a period of time encompassing at least one heartbeat.

In at least one example, the method further includes calculating a volume flow rate time profile in the bodily organ over the period of time encompassing at least one heartbeat.

### BRIEF DESCRIPTION OF THE DRAWINGS

The previous summary and the following detailed descriptions are to be read in view of the drawings, which illustrate particular exemplary embodiments and features as briefly described below. The summary and detailed descriptions, however, are not limited to only those embodiments and features explicitly illustrated.

FIG. 1 shows a flowchart representing a method of determining flow rate, for example of a bodily fluid in a biological conduit, using a velocity vector map according to at least one embodiment. Flow diagrams reference blood flow as a tangible example of a body fluid, hence the reference of theoretical blood flow rate (TBFR) and blood flow rate (BFR).

FIG. 2 shows a flowchart representing a method, according to at least one embodiment, of determining volume flow rate of a bodily fluid using multiple time-incremented velocity maps.

FIG. 3 shows an exemplary velocity vector map and a corresponding velocity component map generated therefrom.

FIG. 4 shows a gradient map generated by applying edge finding analysis to the velocity component map of FIG. 3.

FIG. 5 shows test vectors implemented upon the gradient map of FIG. 4 at angular increments to identify vectors that are approximately perpendicularly across edges.

FIG. 6 shows a test vector optimized to perpendicularly cross a flow path to use in determining vessel diameter according to at least one embodiment.

FIG. 7 shows determined vessel diameter values in a grid array format that matches the resolution and orientation of the velocity vector map of FIG. 3.

FIG. 8 shows cross-sectional areas corresponding to the diameter values of FIG. 7 arranged in a corresponding grid array format.

FIG. 9 shows a volume flow rate map corresponding to the velocity vector map of FIG. 3.

FIG. 10 shows a volume flow rate map serving as a post-procedure example with reference to the volume flow rate map in FIG. 9 as a pre-procedure example.

### DETAILED DESCRIPTIONS

These descriptions are presented with sufficient details to provide an understanding of one or more particular embodiments of broader inventive subject matters. These descriptions expound upon and exemplify particular features of those particular embodiments without limiting the inventive subject matters to the explicitly described embodiments and features. Considerations in view of these descriptions will likely give rise to additional and similar embodiments and features without departing from the scope of the inventive subject matters. Although the term “step” may be expressly used or implied relating to features of processes or methods, no implication is made of any particular order or sequence among such expressed or implied steps unless an order or sequence is explicitly stated.

The following glossary of terms and acronyms may facilitate a better understanding of the descriptions that follow and of the drawings.
Optical Image—Image taken with measurement camera utilizing non laser light (light from additional light source or ambient light.)
High Speed Data Port—USB3, GigE (Gigabit Ethernet), CoaXPress, or higher Network Ports, Frame Grabber cards.
Region of Interest—Area on the investigated tissue to be analyzed for fluid flow rates, usually rectangular in shape.
Speckle response—Optical phenomena resulting from various interactions of coherent laser light on tissue (e.g. refraction, diffraction, reflection, and others).
VVM—Velocity Vector Maps
VCM—Velocity Color Maps
BFR—Body-Fluid or Blood Flow Rate (representing volume per time, e.g.: mL/min or fl.oz./min)
TBFR—Theoretical Body-Fluid or Blood Flow Rate, like BFR but without actual correction via calibrated values
Δ BFR—Delta Fluid or Blood Flow Rate, the mathematical difference of two fluid or blood flow rates, usually before and after surgical intervention of the surgeon (e.g.: Δ BFR=after-earlier)

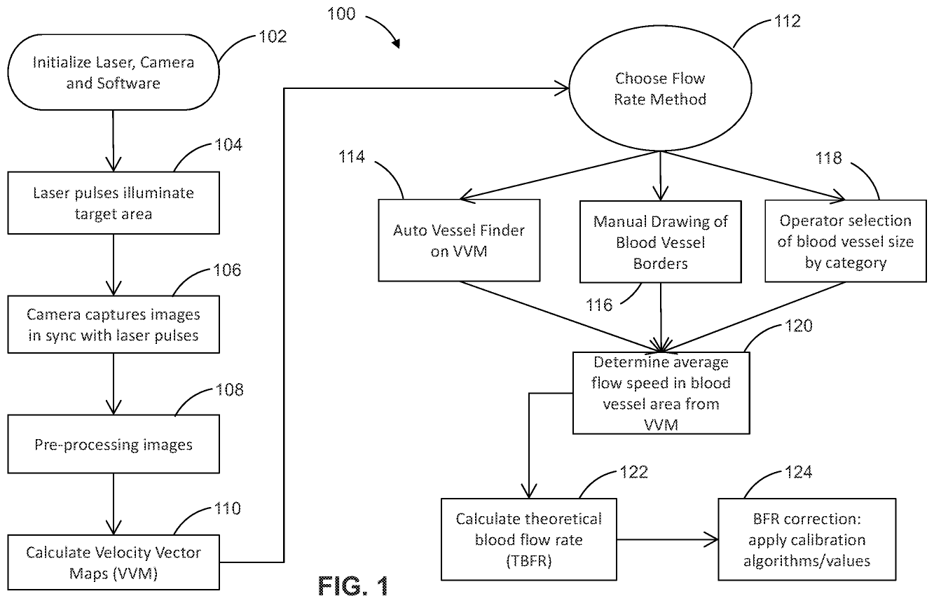

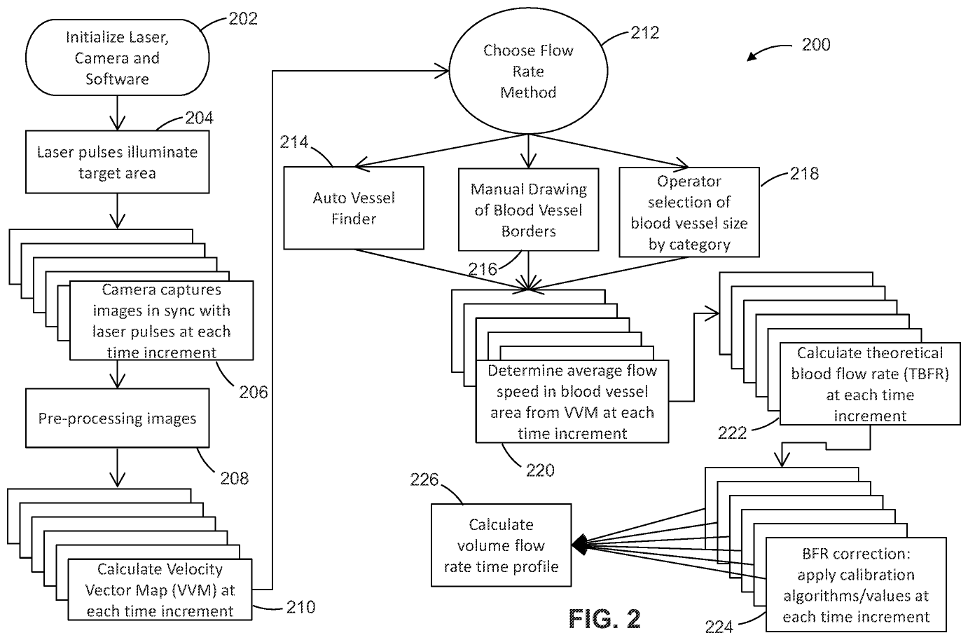

FIG. 1 is a flowchart diagramming a method 100 of determining flow rate, for example of a bodily fluid in a biological conduit, according to at least one embodiment. In the method 100 of FIG. 1, a single velocity vector map may be used in the determination. In the method 200 of FIG. 2, multiple time incremented velocity vector maps are used. These descriptions detail at least one example of how a velocity vector map can be generated. Other ways of generating and collecting velocity vector maps are within the scope of these descriptions. For example, ultrasound and other techniques for generating and collecting velocity vector maps are within the scope of these descriptions.

Biological conduits of interest and subject to analysis according to these descriptions include, but are not limited to: blood vessels such as arteries, capillaries, and veins; digestive tract vessels including those that carry waste fluids such are urine; and other vessels that are walled or otherwise defined flow channels for bodily fluids.

In FIG. 1, a velocity vector map is generated in steps 102 through 110. In step 102, one or more laser(s), one or more camera(s), a data acquisition system and any interfacing modules are initialized. In at least one example, the data acquisition system includes a processing computer in data communication with the laser(s) and camera(s) via a high speed data port(s) and running software. In at least one example, an optical head unit (OHU) houses laser(s) and camera(s) devices, and a control system. The optical head unit is designed to hold the devices in specific alignment and to sink heat to a housing framework or shell

In step 104, the laser(s) pulses illuminate a target area of a test subject, for example upon tissue in the vicinity of a biological conduit in which a bodily fluid flows. In at least one example, a laser(s) is used to produce a speckle response on the exposed tissue. Speckles are thereby created on the subject tissue by the impinging laser(s).

The speckle density may be within a predefined range to provide executable analytical information. Some considerations toward producing a relevant speckle response include: distance from patient; optics for laser beam expansion; and imaging parameters such as lens type, aperture, camera resolution and depth of field. In order to turn a speckle response into relevant images at high-speed, camera wavelength-tuned optical system (illumination and imaging) may be used. To get optimal speckle density, both illumination system parameters and imaging system parameter are optimized and a coded aperture may be applied.

In step 106 (FIG. 1), the camera(s) captures images in synchronization with the laser pulses to capture images of the tissue response to impinging laser. In the example of a laser producing a speckle response, the camera captures high-resolution speckle response images. In at least one example, images are captured via a high speed data port on the processing computer.

As represented by step 108, image processing can start in parallel even as raw data images are stored, for example on a hard-disk. Processing in at least one example consists of several advantageous steps including noise reduction (referring to spurious data signals in data acquisition), background normalization, and contrast improvement. Further image processing, in at least one example, includes identifying area of interest on images, performing fast Fourier transformations to receive 3D spectra on investigated areas, correlating spectra mathematically and reverse Fourier transforming results, and using Gaussian algorithms to identify velocity vectors. In step 110 (FIG. 1), velocity vector maps (VVMs) are produced to represent fluid flow velocities for a given time segment.

In step 112, one of several ways of determining a region of interest in the produced velocity vector map(s) is chosen. Regions of interest represent fluid channels within biological conduits such as vessels in which flow rates can be determined. In these descriptions, three ways of determining a region of interest are detailed, as represented in the flowchart of FIG. 1 by the three branches after the selection made in step 112. Other ways of determining a region of interest are within the scope of these descriptions.

According to at least one embodiment, determining a region of interest in the produced velocity vector map(s), as represented in step 114, includes automatically finding a vessel in one or more VVM, for example by mathematically identifying gradients in the map(s) that represent the walls or boundaries (in two dimensions or 2D) of biological conduits such as vessels in a region of interest, and performing orthogonal vector analysis to retrieve vessel diameter. Assuming that a corresponding (three dimensional or 3D) vessel has a circularly shaped cross section, the total vessel cross-sectional area and/or volume can be calculated as a circular cylindrical tube shaped object. Assuming that a corresponding (three dimensional or 3D) vessel has a rectangular shaped cross section, the total vessel cross-sectional area and/or volume can calculated as a rectangular tube shaped object.

Alternatively, according to at least one other embodiment, determining a region of interest in the produced velocity vector map(s), as represented in step 116, includes an operator identifying vessel borders by drawing lines onto a color map, a vector map image or other image type, for example with a semitransparent overlay of an optical image. Assuming that a vessel shows a circular shaped cross section, the total blood vessel volume is calculated as a tubular shaped object.

In yet another embodiment of determining a region of interest in the produced velocity vector map(s), as represented in step 118, a number of vessels representing a broad range of vessel diameters are shown on a color map image with or without optical image overlays, to let the operator choose the vessel category for vessel total volume identification. For example, assuming that a vessel has a tubular shaped cross section, the total vessel volume is calculated as a cylindrically shaped object.

Other grid-based approaches that may also be used in determining a region of interest in the produced velocity vector map(s), including an example in which a fiducial or pattern of fiducials is projected via a secondary laser illumination system onto the tissue target area, wherein image scaling information may be accomplished by analysis of interfiducial distances. A pattern of multiple fiducials may be used to allow for correction of curvature or non-planarity of the tissue target. When analyzing a vector map, which may be represented in color, against the fiducials, vessel diameters can be estimated by identifying velocity ranges within the areas between fiducials. In another example of a fiducial based approach, a fiducials are displayed as an overlay on a color map or a vector map image to allow the operator to identify areas of interest within vessel regions. Assuming that a vessel shows a circular shaped cross section, the total vessel volume is calculated as a tubular shaped object.

As represented in step 120 in FIG. 1, an average flow speed in a vessel cross-sectional area is determined from the velocity vector map (VVM). Thus, whether a single approach among those represented as steps 114, 116 and 118 is used, or a combination of such approaches is used, a cross-sectional area of a biological conduit is determined using a velocity vector map representing moving entities or moving fluid portions in a bodily fluid flowing within the biological conduit, and an average speed is calculated for the moving entities or moving fluid portions in the bodily fluid flowing across the determined cross-sectional area of the biological conduit.

A theoretical fluid or blood flow rate (TBFR) is calculated as represented in step 122 in FIG. 1. In step 124, TBFR values are corrected, for example using existing calibration data from a database (aligned against a calibration curve, or corrected with a factor) to calculate a fluid or blood flow rate (BFR). Thus, a volume flow rate of a bodily fluid in a biological conduit can be calculated from a determined cross-sectional area and the calculated average speed.

One or more display methods can be used to display results. Plain BFR images can be displayed. As a non-exclusive example, plain BFR images can be represented as optical images with BFR labels pointing towards regions of interest. Comparative images can be displayed. Multiple images of the same region of interest but with different timestamps are displayed in one example. In that example, an earlier image and a later image can be separately displayed. Alternatively, such images can be displayed in overlay format, for example with one or more earlier images displayed overlaying or underlying a later image. Difference images can be displayed. As a non-exclusive example, difference images can be represented as optical images with BFR labels showing BFR values at different timestamps or directly as ΔBFR images.

Fluid flow rate calibrations can be conducted verifying or calibrating BFR values against engineered fluid flow solutions, with a range of vessel diameters and different transportation solutions. Various simulated vessel sizes can be used to facilitate calibration for any type and size of vessel, including vessels with small diameters, such as capillaries (5-10 micrometer), and major vessels, such as the aorta (˜3.0 cm). Measurements can be performed until the TBFR is reached and calibrations can be conducted against actual BFR values, with repeated calibrations for a wide range of flow rates. The generated calibration curves can be stored in a database. Calibration against BFR truth data can be derived by in vivo measurement using alternative means such as Doppler ultrasound, Doppler MRI or via radiographic means.

FIG. 2 is a flowchart diagramming a method 200, according to at least one embodiment, of determining volume flow rate of a bodily fluid in a biological conduit in which the flow of the bodily fluid is continuously variable. In the method 200 of FIG. 2, multiple time-incremented velocity vector maps are used, whereas in FIG. 1, a single velocity vector map may be used in the determination.

In FIG. 2, multiple velocity vector maps are generated in steps 202 through 210. In step 202, one or more laser(s), one or more camera(s), a data acquisition system and any interfacing modules are initialized. In at least one example, the data acquisition system includes a processing computer in data communication with the laser(s) and camera(s) via a high speed data port(s) and running software. In at least one example, an optical head unit (OHU) houses key portions of the illumination and imaging subsystems. The optical head unit is designed to hold the devices in specific alignment with respect to one another and via an arm assembly in specific alignment with the tissue target area. The optical head unit assembly also provides a stable environment to ensure that the illumination and imaging systems operate properly while ensuring that the system is compatible with the appropriate medical interventional environment (e.g., operating room).

In step 204, the laser(s) pulses illuminate a target area of a test subject, for example upon tissue in the vicinity of a biological conduit in which a bodily fluid flows. In at least one example, a laser(s) is used to produce a speckle response on the exposed tissue. Speckles are thereby created on the subject tissue similarly as described with reference to FIG. 1.

In step 206 (FIG. 2), the camera(s) captures time-incremented images in synchronization with the laser pulses to capture images of the tissue response to the impinging laser(s). In the example of a laser producing a speckle response, the camera captures speckle response images. In at least one example, images are captured via a high speed data port on the processing computer. As represented by step 208, image processing can start in parallel even as raw data images are stored, for example on a hard-disk. Processing in at least one example consists of several advantageous steps including noise reduction (referring to spurious data signals in data acquisition), back ground normalization, and contrast improvement. Further image processing, in at least one example, includes identifying area of interest on images, performing fast Fourier transformations to receive 3D spectra for areas of interest, correlating spectra mathematically and Fourier transforming results, and using Gaussian algorithms to identify velocity vectors. In step 210 (FIG. 2), velocity vector maps (VVMs) are calculated for each time increment, each representing fluid flow velocities for a given time segment.

Thus, the method 200 includes collecting, at a frequency of collection, a series of velocity vector maps over time, the velocity vector maps representing moving entities or moving fluid portions in a bodily fluid flowing within a biological conduit. The frequency of collection refers to the inverse of the time increment between the captured time-incremented VVMs.

In step 212, one of several ways of determining a region of interest in the produced velocity vector maps is chosen. As in FIG. 1, three ways of determining a region of interest are detailed, as represented in the flowchart of FIG. 2 by the three branches after the selection made in step 212. Other ways of determining a region of interest are within the scope of these descriptions.

According to at least one embodiment, determining a region of interest in the produced velocity vector map(s), as represented in step 214 (FIG. 2), includes automatically finding a vessel in each VVM as described above with reference to step 114 in FIG. 1.

Alternatively, according to at least one other embodiment, determining a region of interest in the produced velocity vector maps, as represented in step 216 (FIG. 2), includes an operator identifying vessel borders by drawing lines onto color maps or vector map images as described above with reference to step 116 in FIG. 1.

In yet another embodiment of determining a region of interest in the produced velocity vector maps, as represented in step 218 (FIG. 2), a number of vessels representing a broad range of vessel diameters are shown on a color map image with or without optical image overlays, to let the operator choose the vessel category for vessel anatomical definition and total volume identification as described above with reference to step 118 in FIG. 1.

As represented in step 220 in FIG. 1, an average flow speed in a vessel cross-sectional area is determined from the velocity vector map (VVM) of each time increment. Thus, whether a single approach among those represented as steps 214, 216 and 218, or a combination of such approaches is used, a cross-sectional area of the biological conduit can be determined using at least one of the velocity vector maps, and an average speed can be calculated for the moving entities or moving fluid portions in the bodily fluid flowing across the determined cross-sectional area of the biological conduit.

A theoretical fluid or blood flow rate (TBFR) at each time increment is calculated as represented in step 222 in FIG. 2. In step 224, TBFR values are corrected, for example using existing calibration data from a database (aligned against a calibration curve, or corrected with a factor) to calculate a fluid or blood flow rate (BFR) at each time increment. Thus, a volume flow rate of the bodily fluid in the biological conduit can be calculated from a determined cross-sectional area and a calculated average speed.

In step 226, a flow rate profile is calculated representing the fluid or blood flow rate (BFR) values calculated in step 224 over time. The flow rate profile tracks volume flow rate values in the time domain of the collected series of velocity vector maps. For example, the flow rate profile 226 of the bodily fluid can characterize the fluid flow rate as a function of time. The velocity vector maps may be collected in step 210 representing blood flow in a bodily organ over a period of time encompassing heartbeat(s) or other time span of interest. In that example, the flow rate profile is calculated in step 226 over a period of time encompassing a heartbeat or other time span of interest.

To meaningfully characterize the flow of a bodily fluid over a period of time, the frequency of collection of the series of velocity vector maps over time can be chosen to at least satisfy a Nyquist criterion for the flow rate profile of the bodily fluid. The frequency may be selected to be greater than that needed to satisfy a Nyquist criterion for the flow rate profile. For example, to characterize the flow of blood in a bodily organ over a period of time encompassing a heartbeat, a shorter time increment (between the captured time-incremented VVMs) than a time increment needed to satisfy the Nyquist criterion can be selected.

Visual data analytics can be conducted. For example, flow rates across an area of tissue before and after procedures, along with patient outcomes and related metadata (for example based on conventional medical DICOM format) can be normalized across different patients. Normalized images can be data mined to find correlations between patient metadata, specific interventions, and quantified flow pattern changes. Predictive analytics can be performed to help physicians and nurses improve techniques and assumptions and thus improve patient health outcomes.

Furthermore, statistical evaluations of surgery and therapy outcome data (based on patient condition, age, sex, ethnic groups, and other considerations) can help the decision making process during a surgical procedure. For example, the flow rate sufficient for a certain artery in a patient belonging to a certain size or age group can be determined. For patient information privacy and security, raw data may not be publicly accessible, and data may be cleaned of patient information. Gesture controlled processing may be implementing, for example to permit surgeons to navigate and control images and data processing without cross contaminating other hardware interfaces and surgical gloves during procedures.

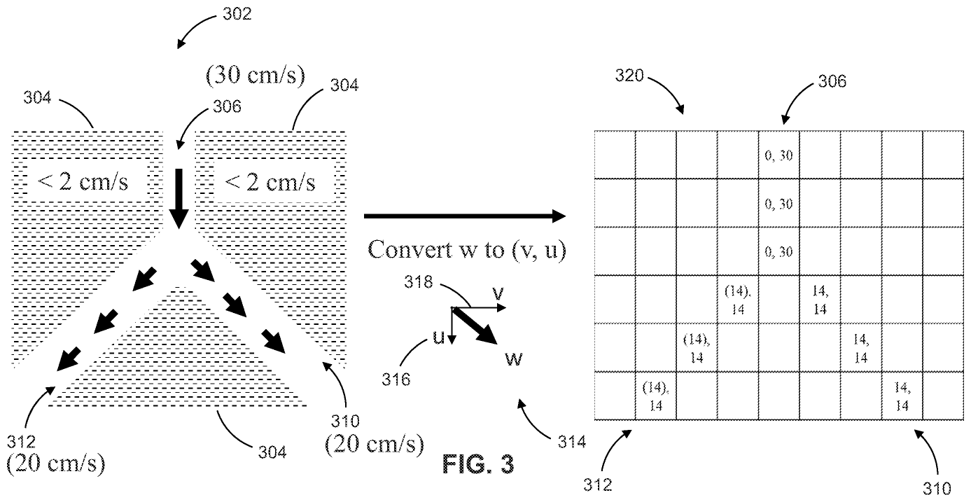

FIGS. 3-9 further illustrate the determination of flow rates in regions of interest, for example that of bodily fluids within biological conduits, from a velocity vector map. An exemplary velocity vector map 302 is shown as a two dimensional image in FIG. 3 and represents various data objects in various formats including matrices and other data arrays that contain velocity data corresponded with pixels representing locations. The hatched areas 304 represent regions in which little or no velocity is measured or represented, for example as measured in the illustrated example as below two centimeters per second. Higher velocities are measured or represented in three flow zones 306, 310 and 312. In the illustrated example, the flow zone 306 has a measured or represented velocity value of thirty centimeters per second, and the flow zones 310 and 312 each has a measured or represented velocity value of twenty centimeters per second. The flow zone 306 in at least one example represents an entry vessel by which blood or other biological fluid enters the region of the velocity vector map 302, whereas the flow zones 310 and 312 represent exit vessels, branching from the entry vessel, that carry the blood or fluid from the region.

A vector diagram 314 represents a U axis 316 and a V axis 318 corresponding to respective directions in the velocity vector map 302 illustrated as down and to the right in FIG. 3. That is, the flow zone 306 has a vector velocity of thirty centimeters per second directed along the U axis 316. The flow zone 310 has a vector velocity directed approximately forty five degrees between the V axis 318 and the U axis, and has flow components of approximately fourteen centimeters per second along the U and V axes. The flow zone 312 has a vector velocity directed approximately forty five degrees between the negative of the V axis and the U axis, and also has U and V flow magnitudes of approximately fourteen centimeters per second. An arbitrary vector W can be specified by or broken into its U and V components.

An exemplary U and V component map 320 is generated by conversion of the velocity vector map 302 into a grid format in which each pixel has U and V velocity components, illustrated with negative sign values in parentheses. Grid locations of pixels of any size can be selected. For example, in the U and V component map 320 the flow zone 306 appears as a single pixel column.

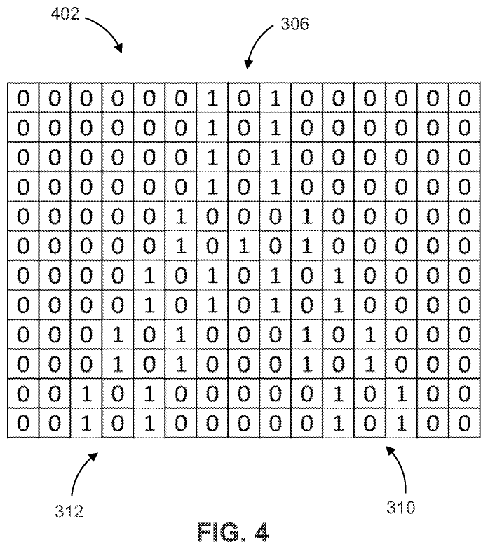

Edge finding analysis is applied to the U and V component map 320 to generate the gradient map 402 of FIG. 4. For example, significant changes in two dimensional and three dimensional gradients of values can be found automatically and enumerated as a matrix of values such as ones and zeroes as shown in the gradient map 402. Functionality of available analytical tools such as MATLAB may facilitate or implement gradient mapping. In the illustrated example, values of one are found at pixels or locations of abrupt change relative to nearest neighbors. Thus boundaries of the flow zones 306, 310, and 312 of the velocity vector map 302 of FIG. 3 are represented as lines of pixels having values of one in the gradient map 402 of FIG. 4, which is represented in higher resolution than the velocity vector map 302 and the U and V component map 320 of FIG. 3.

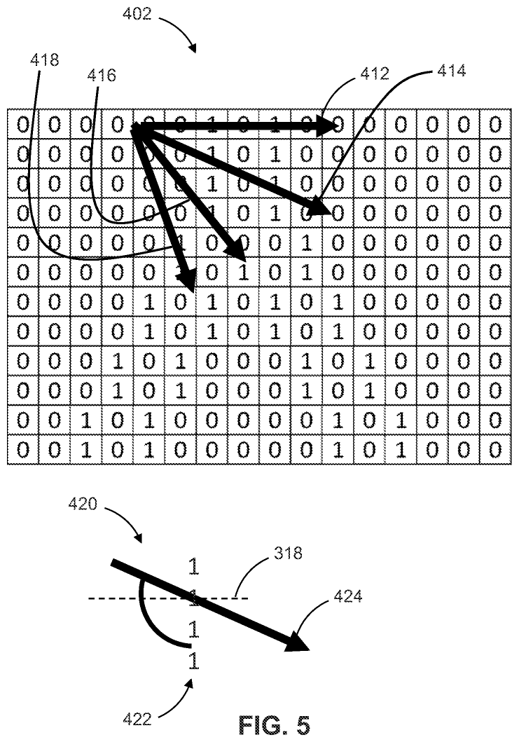

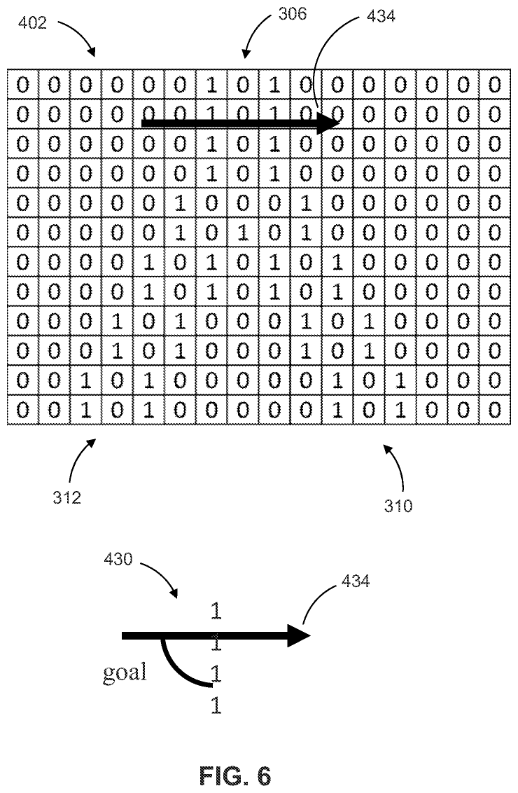

The determination of edges of flow zones, using the gradient map 402, so as to determine dimensions such as spans, diameters, and cross-sectional areas of the flow zones is represented in FIGS. 5 and 6. As represented in FIG. 5, test vectors are implemented upon the gradient map 402 at angular increments, for example starting at zero degrees (at the V axis 318) and rotating to three hundred and sixty degrees by increments of six degrees. Four representative incremented test vectors 412, 414, 416 and 418 are shown to illustrate the approach. The test vectors that are perpendicular to the lines of pixels having values that equal one in the gradient map 402 are identified. The lengths of segments along those identified vectors are determined as the distances between nearby pixels having values of one, representing nearby lines of one-valued pixels and thus the edges of flow zones corresponding to biological conduits such as blood vessels in at least one example. The determined lengths may range as approximately one to three times the diameter of a vessel in a biological specimen for which the velocity vector map 302 (FIG. 3) and the corresponding gradient map 402 were generated. This error or tolerance range is due to poor alignment of the roughly identified test vectors relative to a perpendicular direction across a column 422 of one-valued pixels as represented by the identified test vector 424 in the plot 420 representing the rotation of test vectors in FIG. 5.

As represented in FIG. 6, iterations at finer angular increments around identified test vectors can be implemented upon the gradient map 402 to determine more optimized vector solutions and more reliable and accurate measurements along those optimized vectors. The goal for optimization is identifying vectors that are perpendicular across lines of one-valued pixels, as represented in the plot 430 in FIG. 6 by the optimized vector 434. The lengths of segments along those optimized vectors are determined as the distances between nearby pixels having values of one, which represent nearby lines of one-valued pixels and thus the edges of flow zones corresponding to biological conduits such as blood vessels in at least one example. A length so determined represents the measured span or diameter of a vessel in a biological specimen for which the velocity vector map 302 (FIG. 3) and the corresponding gradient map 402 was generated.

In the illustrated example, the optimized vector 434 is used to determine the diameter of a vessel represented by the flow path 306. The diameters of the vessels represented by the flow paths 310 and 312 are similarly determined using respective optimized vectors.

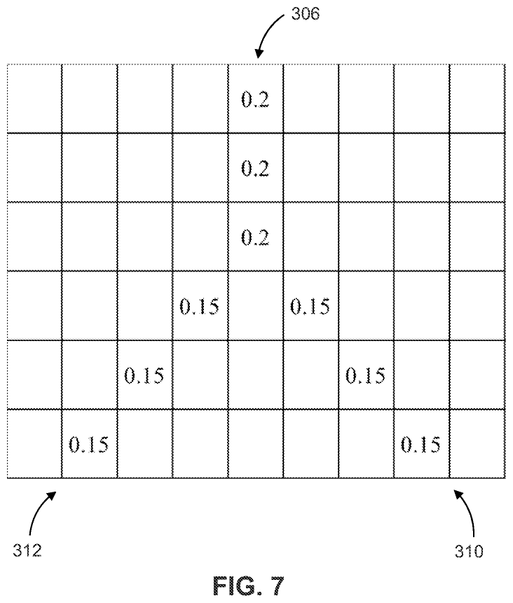

As represented in FIG. 7, the diameters as determined with reference to FIG. 6 are shown in a grid array format that matches the resolution and orientation of the velocity vector map 302 of FIG. 3. The non-zero values along the flow paths 306, 310 and 312 provide the determined diameters of the flow paths. The units for the values in FIG. 7 are centimeters but the methodology may be employed across any length scale.

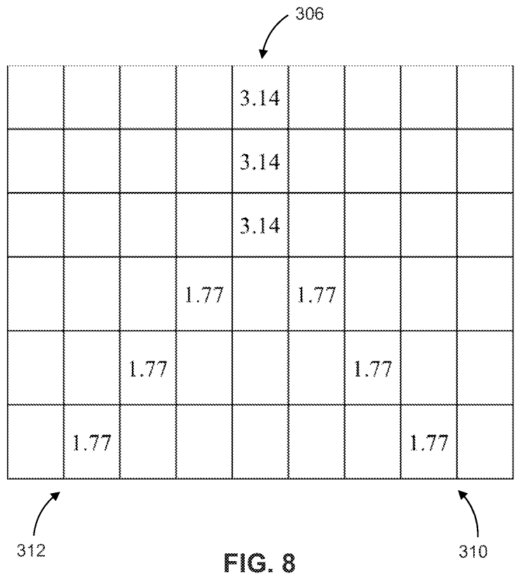

As represented in FIG. 8, cross-sectional areas corresponding to the diameters of FIG. 7 are arranged in a corresponding grid array format. The units for the values in FIG. 8 are 0.01 squared centimeters. Assuming that vessels are circular in cross section, cross-sectional area is calculated as:

Area=π×(Diameter/2)²

Assuming a tubular three dimensional geometry, the correspondence among small volume flow rate and area and speed (Length/Time) is expressed as:

Volume Flow Rate=Area×Length/Time

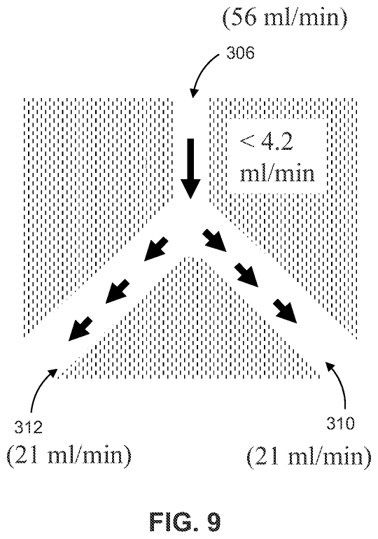

Speed (Length/Time) is taken from the velocity vector map 302 of FIG. 3. Thus multiplying the values in the velocity vector map 302 by the values in the cross-sectional area map of FIG. 8, for example on a pixel by corresponding pixel basis, Volume Flow Rate values are calculated and presented in FIG. 9, with values converted from units of centimeters cubed per second to milliliter per minute corresponding to conventional units by which flow rates for blood and other biological fluids are typically measured or considered.

FIG. 9 shows a volume flow rate map corresponding to the velocity vector map of FIG. 3. FIG. 9 thus provides volume flow rates corresponding to the velocity vector map 302 of FIG. 3. As such, the volume flow rates for the three flow paths 306, 310 and 312 are provided.

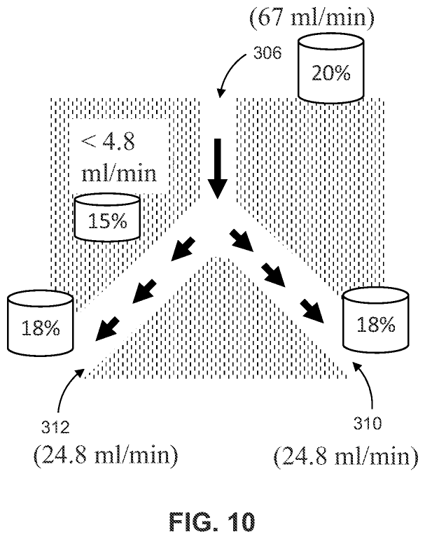

FIG. 10 shows a volume flow rate map serving as a post-procedure example with reference to the volume flow rate map in FIG. 9 as a pre-procedure example. Flow rate percentage increases are shown for each area within cylinders, or other convenient objects, and having heights that vary roughly proportional to the relative change in fluid flow in that area. In this example, FIG. 10 is generated after a procedure, which could be medical and/or surgical, that increases flow rates by the percentages shown.

Particular embodiments and features have been described with reference to the drawings. It is to be understood that these descriptions are not limited to any single embodiment or any particular set of features, and that similar embodiments and features may arise or modifications and additions may be made without departing from the scope of these descriptions and the spirit of the appended claims.

What is claimed is:

1. A method of determining volume flow rate of a bodily fluid in a biological conduit, the method comprising:
determining a cross-sectional area of a biological conduit using a velocity vector map representing moving entities or moving fluid portions in a bodily fluid flowing within the biological conduit;
calculating an average or instantaneous speed of the moving entities or moving fluid portions in the bodily fluid flowing across the determined cross-sectional area of the biological conduit; and
calculating volume flow rate of the bodily fluid in the biological conduit from the determined cross-sectional area and the calculated average speed.

2. The method of claim 1, wherein determining the cross-sectional area of the biological conduit comprises determining edges of the biological conduit.

3. The method of claim 2, wherein determining edges of the biological conduit comprises identifying edges in the velocity vector map.

4. The method of claim 3, wherein determining edges of the biological conduit comprises determining gradients.

5. The method of claim 2, wherein determining edges of the biological conduit comprises a user determining the edges viewing a display of the velocity vector map.

6. The method of claim 1, wherein determining the cross-sectional area of the biological conduit comprises generating a contour map.

7. The method of claim 6, wherein determining the cross-sectional area of the biological conduit comprises identifying parallel line segments in the contour map.

8. The method of claim 1, wherein determining the cross-sectional area of the biological conduit comprises determining a width of the biological conduit and assuming the width represents a diameter.

9. The method of claim 1, wherein determining the cross-sectional area of the biological conduit comprises mathematically identifying borders of the biological conduit and performing orthogonal vector analysis to retrieve a diameter.

10. The method of claim 1, further comprising receiving indication of a user choice of vessel category according to fluid vessel volume.

11. The method of claim 1, further comprising receiving indication of a user choice of vessel category according to biological tissue type.

12. The method of claim 1, further comprising receiving indication of a user choice of vessel category according to anatomical classification.

13. The method of claim 1, wherein calculating volume flow rate comprises calculating a theoretical body fluid or blood flow rate (TBFR) value, and correcting the TBFR value with calibration data from a database.

14. A method of determining volume flow rate of a bodily fluid in a biological conduit wherein the flow of the bodily fluid is continuously variable, the method comprising:
collecting, at a frequency of collection, a series of velocity vector maps over time, the velocity vector maps representing moving entities or moving fluid portions in a bodily fluid flowing within a biological conduit;
determining a cross-sectional area of the biological conduit using at least one of the velocity vector maps;
calculating an average speed of the moving entities or moving fluid portions in the bodily fluid flowing across the determined cross-sectional area of the biological conduit; and
calculating volume flow rate of the bodily fluid in the biological conduit from the determined cross-sectional area and the calculated average speed.

15. The method of claim 14, wherein the frequency of collection satisfies a Nyquist criterion for a flow rate profile of the bodily fluid.

16. The method of claim 14, wherein the frequency of collection is greater than a Nyquist criterion for a flow rate profile of the bodily fluid.

17. The method of claim 14, further comprising calculating a volume flow rate profile.

18. The method of claim 14, wherein calculating an average speed comprises calculating an average speed from multiple velocity vector maps of the series of velocity vector maps.

19. The method of claim 14, wherein collecting a series of velocity vector maps over time comprises collecting a series of velocity vector maps representing blood flow in a bodily organ over a period of time encompassing at least one heartbeat or other arbitrary time frames.

20. The method of claim 19, further comprising calculating a volume flow rate time profile in the bodily organ over the period of time encompassing at least one heartbeat.
